(19) United States
(12) Patent Application Publication
(10) Pub. No.: US 2023/0337978 A1
(43) Pub. Date: Oct. 26, 2023

(54) DEVICE WITH DIELECTRIC MATERIAL TO OPTIMIZE MAGNETIC RESONANCE IMAGING

(71) Applicants: Sung-Min Sohn, Chandler, AZ (US); Sri Kirthi Kandala, Tempe, AZ (US); Seyedamin Hashemi, Tempe, AZ (US)

(72) Inventors: Sung-Min Sohn, Chandler, AZ (US); Sri Kirthi Kandala, Tempe, AZ (US); Seyedamin Hashemi, Tempe, AZ (US)

(21) Appl. No.: 18/305,182

(22) Filed: Apr. 21, 2023

Related U.S. Application Data
(60) Provisional application No. 63/334,015, filed on Apr. 22, 2022.

(51) Int. Cl. A61B 5/00; G01R 33/387; A61B 5/055

(52) U.S. Cl. CPC A61B 5/6803; A61B 5/0042; A61B 5/055; G01R 33/387; A61B 2562/12; A61B 2562/168

(57) ABSTRACT

A dielectric pad and method of fabricating a dielectric pad for improving signal-to-noise ratio and image quality in MRI procedures. The dielectric pad includes a composition of TiO₂, BaTiO₃, or SiC mixed with a solvent to produce a flexible and stretchable pad for MRI use. The flexible and stretchable pad is configured to conform to various body shapes, to be wrap-able, and to be wearable for MR imaging procedures.

### CROSS REFERENCE TO RELATED APPLICATIONS

This application is a non-provisional of U.S. Provisional Pat. Application No. 63/334,015, filed on Apr. 22, 2022, the entire contents of which are incorporated herein by reference.

### BACKGROUND

Magnetic resonance imaging (MRI) is one of the most powerful non-invasive tools to view anatomical structures and diagnose medical conditions. Modern MRI scanners use high static magnetic fields and multi-channel radiofrequency (RF) technology to obtain better anatomical images. The use of higher static magnetic field (B₀) strength can provide higher signal-to-noise ratio (SNR), temporal resolution, and spatial resolution. However, there is a critical challenge to overcome in that a higher B₀ field results in faster Larmor precession, thus resulting in a shorter wavelength. This shorter wavelength results in local RF field (B₁) inhomogeneity, which degrades the quality of the acquired images. Although employing parallel RF transmission with multichannel coils and the use of multichannel receive coils being widely studied to mitigate the transmit RF field (B₁⁺) inhomogeneity problem, costly upgrades and advances in an entire MRI system are necessary.

As an alternative, dielectric materials are often used in MRI to increase and unify the SNR in a large field of view (FOV). Dielectric pads effectively act as local B₁ field shimming devices and reduce the specific absorption rate (SAR) of the sample. It has been shown that SAR is directly related to the induced RF displacement currents within the dielectric material by transforming RF electric fields into RF magnetic fields. Additionally, these materials have shown to be effective in enhancing the B₁ field strength, especially in the areas with reduced B₁ field penetration caused by using high frequency. Various forms of dielectric materials are used such as dilute solutions, slurries, gels, and solid ceramics in the manner of pads, helmets, and discs. Studies with these dielectric materials using ferroelectric ceramic particles such as barium titanate (BaTiO₃), calcium titanate (CaTiO₃), and titanium dioxide (TiO₂) show that the B₁ field distribution can be manipulated with a strong dependence on the dielectric constant of the material. Previously demonstrated, a condensed solid of these materials can have a high dielectric constant (εᵣ > 1000).

There are some challenges in using conventional dielectric materials. A slurry pad and a dielectric helmet filled with a solution have a risk of liquid leaks. On the other hand, a rigid helmet cannot conform to different head sizes. Local dielectric materials can focus the B ₁ field on a specific region as an RF shimming, but it tends to decrease the B₁ field in other parts because of RF electromagnetic (EM) sensitivity, and it is challenging to wrap a sample with local pads or discs to evenly keep the effect of dielectric material. The variable thicknesses and distances between media (e.g., air, metallic conductors, dielectric materials, and a sample) results in heterogeneous EM boundary conditions that increase the complexity of modeling and inhomogeneous B₁ penetration. In addition to all these challenges, some materials are visible in MR images. Thus, they may cause chemical shifts. They may also create difficulties when setting up the homogeneous main magnetic field in the shimming process because of the inclusion of precessing protons in the dielectric materials. Finally, it has been reported that chemical structures age over time. Evaporation of a solution, oxidation, and sensitivity to humidity and temperature result in significant variation of dielectric properties over their lifetime, hence degrading their efficiency.

Recent studies concerning the employment of dielectric pads for enhancing MR images have been widely investigated, with the aim to find the best-performing size and shapes for these pads. However, the challenge of finding the best structure has not been precisely addressed. The major challenges include: (1) proper pad positioning is critical for improving field homogeneity but it is difficult to predict how the position will affect images, and pad adjustment after imaging has commenced is challenging, (2) the use of dielectric pads with different shapes, and the presence of spaces between mediums (e.g., an RF coil, a dielectric pad, and a subject) increase an arbitrary RF field distribution because of heterogeneous RF boundary conditions, and (3) the fixed electrical properties of dielectric pads are difficult to adapt to different RF loading conditions. Therefore, the use of dielectric pads is currently suboptimal.

Thus, it is desirable to develop more predictable and tunable dielectric materials for clinical use.

### SUMMARY

Dielectric materials can improve image quality when placed on the body surface to increase the uniformity and strength of the B₁ field propagation. In general, several high-dielectric pads are placed between an RF coil and a subject to aid in uniformly increasing RF transmission into the subject.

The present disclosure provides a flexible, stretchable, and MR invisible dielectric material that uniformly increases the local magnetic field (B₁) in MRI. A novel technique is disclosed addressing the use of high dielectric constant, visible, formless, and inflexible pads. The present disclosure describes (i) making pads conforming to different shapes and sizes of subjects, (ii) decreasing the EM sensitivity, (iii) reducing the inhomogeneity caused by the movement of the pad, and (iv) eliminating dielectric materials artifacts due to their visibility.

The present disclosure also provides a wearable, inflatable, and tunable dielectric material for use in MRI. The wearable dielectric material is tight-fitting and comprises a mixture of dielectric particles and stretchable polymer. The wearable dielectric material reduces the irregularity and variables to make the most of dielectric materials. The wearable material includes empty spaces or cavities that expand or contract using a liquid-based dielectric solution using a pneumatic liquid delivery system. Tuning of the dielectric material is adjusted by a wireless user interface to address different conditions during the scan.

In one implementation, the disclosure provides a wearable device to optimize images in a 7T MRI system. The device comprises a cap wearable on a head-shaped phantom, the cap comprising a mixture of dielectric particles and a stretchable polymer.

In one embodiment, the present disclosure provides a dielectric pad comprising an elastomeric material, and a dielectric material dispersed in the elastomeric material, the dielectric material comprising TiO2, BaTiO3, or SiC, or a combination thereof. The dielectric pad has a dielectric constant of at least 10.

In another embodiment, the present disclosure provides a method of fabricating a dielectric pad for magnetic resonance imaging. The method comprises mixing a dielectric material with an elastomer to form a solution, pouring the solution into a mold, applying a vacuum to the solution to remove air bubbles, and maintaining the solution at room temperature.

In a further embodiment, the present disclosure provides a device to optimize images of a target acquired by MRI scanner. The device comprises a cap wearable on a head, the cap comprising a mixture of a dielectric material and an elastomeric material, the cap configured to inflate and deflate to optimize MR images acquired by the MRI scanner.

Other aspects of the present disclosure will become apparent by consideration of the detailed description and accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A schematically illustrates a fabrication process to make a dielectric pad according to an embodiment of the present disclosure.

FIG. 1B illustrates flexibility and stretchability of a fabricated dielectric pad according to an embodiment of the present disclosure.

FIG. 2 illustrates full wave electromagnetic simulation results. (A) illustrates the dielectric material models used in the simulation; (B) illustrates |B₁| field distribution without a dielectric pad; (C) illustrates an average SAR map without a dielectric pad; (D) illustrates |B₁| field distribution with different (flat, curved, and wrapped) configurations and dielectric constants (25, 50, and 100); and (E) illustrates average SAR maps with the different configurations and dielectric constants.

FIG. 3 illustrates a noise analysis and MRI compatibility evaluation in phantom imaging. (A) is an image of a small cylindrical phantom without any dielectric pad as a reference; (B) is an image of the cylindrical phantom with multiple layers of a purely Ecoflex-based pad; (C) is an image of the cylindrical phantom with a BaTiO₃-based dielectric pad; (D) is an image of the cylindrical phantom with a TiO₂-based dielectric pad; and (E) is an image of the cylindrical phantom with a SiC-based dielectric pad wrapped around the phantom.

FIG. 4 illustrates signal intensity and uniformity analyses in phantom imaging. (A) illustrates a schematic configuration of the cylindrical phantom, dielectric pad, and receive coil relative to each other; (B) illustrates an image of the cylindrical phantom without any dielectric pad (reference); (C) illustrates images of a single layer of purely Ecoflex-based pad (top), a purely Ecoflex-based small patch (bottom); (D) illustrates images of a BaTiO₃-based pad (top), a BaTiO₃-based small patch (bottom); (E) illustrates images of a TiO₂-based pad (top), and TiO₂-based small patch (bottom) wrapped around the phantom. (F) graphically illustrates the signal intensity plot along the dotted line shown in top of schematic (A) of pads completely covering the phantom. (G) graphically illustrates the signal intensity plot along the dotted line shown in bottom of schematic (A) of small patches covering just a part of phantom.

FIG. 5 illustrates evaluation of dielectric pads in oxtail imaging. (A) illustrates an oxtail image without using any dielectric pads; (B) illustrates an image of a single layer of a purely Ecoflex-based pad; (C) illustrates an image of a BaTiO₃-based dielectric pad; and (D) illustrates an image of an invisible TiO₂-based dielectric pad wrapped around the oxtail.

FIG. 6 (a) illustrates EM simulation at 7T with varying dielectric constants; (b) photographs of a dielectric helmet; and (c) photograph of an artificial EM material.

FIG. 7 illustrates a schematic diagram of a wearable, inflatable, and tunable dielectric cap for human brain imaging at 7T according to the present disclosure.

FIG. 8 illustrates preliminary study results: (a) MR images without and with dielectric mixtures at a preclinical 7T, (b) a miniaturized head phantom and a wearable dielectric cap for MR images, and (c) the uniformity comparison result.

FIG. 9 illustrates (a) a pneumatic control system with a miniaturized head phantom and an inflatable head cap, (b) before inflation, and (c) after inflation.

FIG. 10 illustrates (a) brain imaging with (left) and without (right) water pad; (b) potential air pocket problem with a dielectric solution; and (c) concept diagram of the tunable dielectric cap.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the embodiments described herein. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not. Further, it should further be noted that the terms “first,” “second,” and the like herein do not denote any order, quantity, or relative importance, but rather are used to distinguish one element from another.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. All ranges disclosed herein are inclusive of the endpoints, and the endpoints are independently combinable with each other. Each range disclosed herein constitutes a disclosure of any point or sub-range lying within the disclosed range.

As used herein, the terms “providing”, “administering,” and “introducing,” are used interchangeably herein and refer to the placement of the compositions of the disclosure into a subject by a method or route which results in at least partial localization of the composition to a desired site. The compositions can be administered by any appropriate route which results in delivery to a desired location in the subject.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

As used herein, “treat,” “treating” and the like mean a slowing, stopping or reversing of progression of a disease or disorder when provided a composition described herein to an appropriate control subject. The terms also mean a reversing of the progression of such a disease or disorder to a point of eliminating or greatly reducing the cell proliferation. As such, “treating” means an application or administration of the compositions described herein to a subject, where the subject has a disease or a symptom of a disease, where the purpose is to cure, heal, alleviate, relieve, alter, remedy, ameliorate, improve or affect the disease or symptoms of the disease.

While the embodiments have been described with reference to preferred embodiments, it will be understood by those skilled in the art that various changes may be made and equivalents may be substituted for the elements thereof without departing from the scope of the embodiments. In addition, many modifications may be made to adapt the teaching of the embodiments described herein to particular use, application, manufacturing conditions, use conditions, composition, medium, size, and/or materials without departing from the essential scope and spirit of the embodiments described herein.

While various aspects and embodiments have been disclosed herein, other aspects and embodiments will be apparent to those skilled in the art. The various aspects and embodiments disclosed herein are for purposes of illustration and are not intended to be limiting of the true scope of the embodiments disclosed herein. It is also to be understood that the terminology used herein is for the purpose of describing particular embodiments only, and is not intended to be limiting. Since many modifications, variations, and changes in detail can be made to the described examples, it is intended that all matters in the preceding description and shown in the accompanying figures be interpreted as illustrative and not in a limiting sense.

All methods described herein can be performed in any suitable order unless otherwise indicated herein or otherwise clearly contradicted by context. The use of any and all examples, or exemplary language (e.g., “such as”), is intended merely to better illustrate the embodiments described herein and does not pose a limitation on the scope of any embodiments unless otherwise claimed.

The present disclosure provides a process to fabricate a stretchable dielectric pad. The EM simulation setup and the MR imaging parameters to acquire images for various samples and pad configurations are discussed below.

Various studies with ferroelectric ceramic particles such as barium titanate (BaTiO₃), calcium titanate (CaTiO₃), or titanium dioxide (TiO₂) show a strong dependence on high dielectric constants in the B₁ fields. A high density of these materials increases the dielectric constant, and a solid bulk can have an ultrahigh level (εᵣ > 1000). However, there is an issue with the use of very or ultra-high dielectric materials. They cannot fully mitigate the inhomogeneity in MR images because the sensitivity of RF interferences is getting higher with higher dielectric constants.

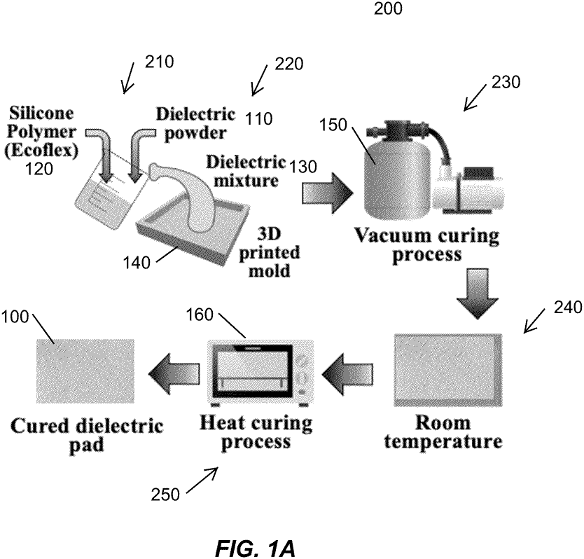

FIG. 1B illustrates a dielectric pad 100 according to an embodiment of the present disclosure. The dielectric pad 100 comprises a composition 110 and a solvent 120. In some implementations, the composition 110 comprises a first dielectric powder (e.g., barium titanate powder (BaTiO₃)), a second dielectric powder (e.g., titanium dioxide powder (TiO₂)), or a third dielectric powder (e.g., silicon carbide powder (SiC)). In some implementations, the solvent 120 comprises an elastic polymer (e.g., a biocompatible silicone elastomer, such as ecoflex®). In some implementations, the dielectric pad 100 comprises a high weight to weight concentration of TiO₂ powder, BaTiO₃, or SiC powder. In one implementation, the dielectric pad 100 comprises 20% by weight of TiO₂. In another implementation, the dielectric 100 comprises 55% by weight of BaTiO₃.

The present disclosure provides a method 200 of fabricating a dielectric pad 100 for a MRI procedure according to an embodiment. In one implementation, as illustrated in FIG. 1A, the composition 110 is mixed with the solvent 120 to produce a flexible and stretchable pad for MRI use. As shown in FIG. 1B, the flexible and stretchable pad is configured to conform to various body shapes, to be wrap-able, and to be wearable for MR imaging procedures.

With reference to FIG. 1A, initially, the composition 110 (e.g, TiO₂ powder, the BaTiO₃ powder, or the SiC powder) is mixed (at 210) with the solvent 120 (e.g., silicone polymer, such as ecoflex). After mixing the TiO₂ powder, the BaTiO₃ powder, or the SiC powder in the ecoflex solvent, the solution 130 is poured (at 220) into a three-dimensional (3D) printed mold 140 and placed (at 230) in a vacuum chamber 150 for a period of time (e.g., 5 minutes) to eliminate air bubbles. The mold 140 is removed (at 240) from the vacuum chamber 150 where the solution remains at room temperature (25° C.) for a period of time (e.g., 10 minutes). The mold 140 is then optionally placed in an oven 160 to speed up the curing process. After curing, the pad 100 is removed from the mold 140 and results in a flexible and stretchable pad 100 as illustrated in FIG. 1A. The dielectric pad has a dielectric constant of at least 10.

### Example 1 - Fabrication of Dielectric Pad for MRI

### Fabrication Process for Stretchable Dielectric Materials

Barium titanate, titanium dioxide, and silicon carbide microscale powders were used with a biocompatible silicone elastomer (Ecoflex, Smooth-On, USA) to fabricate stretchable pads with a high weight to weight concentration of TiO₂ (e.g., 20%), BaTiO₃ (e.g., 55%), and SiC (e.g., 58%) powders. After mixing dielectric powders in the Ecoflex solvent, the solution was placed in a vacuum chamber for 5 minutes to eliminate air bubbles. This solution was poured into a 3D printed mold and remained at room temperature (25° C.) for 10 minutes. Later, the mold was placed in an oven to speed up the curing process. FIG. 1 (at B) shows the flexibility and stretchability of the fabricated pad that can be body conformal, wrap-able, and wearable for MR imaging.

### Numerical Simulation

A full-wave EM simulation was performed with the HFSS Electronics Desktop (e.g., available from ANSYS, Inc. Canonsburg, PA, USA). An eight-leg birdcage coil with bandpass configuration was used as a transceiver with an ellipsoidal phantom that has the electrical characteristics of the average brain tissue with bulk conductivity of εᵣ = 51.89 and permittivity of σ = 0.5528 at 7T. In order to make similar conditions between the EM simulation and the imaging test, the birdcage coil was measured and modeled in HFSS with a 74 mm diameter and 150 mm length.

The RF coil was loaded with the phantom and impedance matched to 300 MHz. The dielectric pads were modeled with 3 mm thickness and different dielectric constants for each simulation. Flat, curved, and wrapped dielectric pad conditions were simulated and their respective B₁ fields were compared with a reference simulation with no dielectric material. A single RF feeding port and an isotropic phantom model were used to rule out other variables in the B₁ field distribution by the effect of dielectric material models.

### Image Acquisition

The 7T MR imaging experiments were performed with a preclinical 7T, 30-cm horizontal-bore magnet and a BIOSPEC® Avance III spectrometer (available from Bruker, Billerica, MA) with 116-mm high-power gradient (600 mT/m). Three different experiments were conducted to evaluate the performance of the dielectric pads. A fast-low-angle-shot (FLASH) sequence was employed with a 5.4 ms echo time (TE) in all experiments and other conditions were the same except for pad configurations in each experiment, respectively. The FLASH sequence used 400 W of peak power, and the Bruker console was used to perform the impedance matching of the transmit coil. Two isotropic phantoms (A and B of 2.85 cm and 5 cm in diameter, respectively) were utilized to analyze the performance of the dielectric pads in terms of additional noise, B₁ field uniformity, and intensity. A medium size oxtail of 5.8 cm in diameter was imaged to understand the impact of dielectric pads on a heterogenous sample closely representing human tissue. The repetition time (TR) was 350 ms for both the oxtail and phantom B, but 766 ms for the phantom A. The chosen flip angle (α) was 20° for the oxtail and phantom B experiments, but 40° for phantom A study. An in-plane resolution of 312 × 312 mm², 234 × 234 mm², and 195 × 195 mm² were achieved with an FOV of 80 × 80 mm², 60 × 60 mm², and 50 × 50 mm² in oxtail, phantom B, and phantom A imaging respectively. 5, 5, and 15 slices with a 1 mm slice thickness were acquired along the oxtail, phantom B, and phantom A, respectively. During each of the studies, the samples were carefully aligned with the same orientation and position with respect to the coil to prevent additional variables from affecting the image.

The first study used a quadrature Bruker transceiver coil with multiple wraps of dielectric pad around a phantom A to evaluate the MR compatibility (e.g., invisibility), and additional noise by the pads. In the second study for the B₁ field analysis, a single channel Bruker volume transmit coil was used with an in-house designed surface loop coil (5 cm diameter). The loop coil was tuned and matched using a vector network analyzer (e.g., FIELDFOX® N9923A, available from Keysight Technologies, USA) for each imaging test. MR images were acquired by wrapping a single layer of dielectric material around the phantom B. Also, images with a small pad were obtained to compare effects of a local patch and an entirely wrapped condition. Mean signal intensity was calculated from the obtained MR images covering the whole area of the phantom and image uniformity was calculated using a percent image uniformity (PIU) equation defined in equation 1:

$$P I E = 100 \times \left[1 - \frac{\left({S}_{m a x} - {S}_{m i n}\right)}{\left({S}_{m a x} + {S}_{m i n}\right)}\right] \qquad \text{(Eq.1)}$$

Where Sₘₐₓ is the average signal intensity in a small region of interest (ROI) chosen from the area of maximum pixel intensity and Sₘᵢₙ is the average signal intensity in a small ROI chosen from the area of minimum pixel intensity. A third study with an oxtail sample was performed with the same coil configuration as study one, to observe the improvement in contrast, MR invisibility of TiO₂ pad, chemical shift, and SNR for a general application with heterogenous samples.

The results indicate that the images from different MR studies are consistent with HFSS EM simulations and show the feasibility of the new flexible, stretchable, and MR invisible dielectric material for easily adoptable clinical applications.

### Simulation

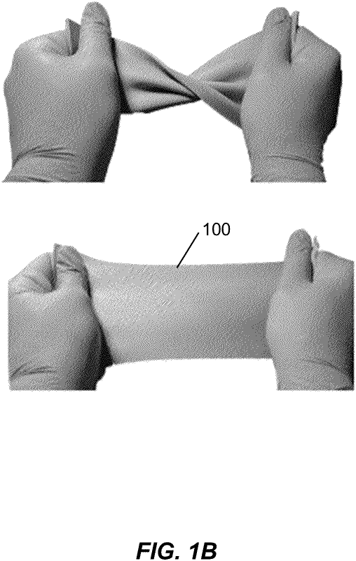

Three configurations of the dielectric pad were modeled in HFSS as shown in FIG. 2 (at A), with three different dielectric constant values for each configuration. The pads were tested in various ways: (1) completely wrapped around the phantom, (2) curved along the phantom to cover a local area at the RF feeding port, and (3) positioning a flat patch shape without wrapping or curvature. A reference B₁ field distribution, per square root of input power, and average SAR in watt per kilogram (W/kg) was generated in the transverse plane of the phantom without any dielectric material as shown in FIG. 2 (at B) and 2 (at C), respectively. The dielectric constants (εᵣ) used for the pads were εᵣ = 25, εᵣ = 50, and εᵣ = 100. Each εᵣ value was simulated for all the three configurations of dielectric pads and their respective B₁ field results are displayed in FIG. 2 (at D). Wrapping the pad around the sample lead to higher B₁ efficiency than the two other pad configurations, and the coil performance was enhanced by increasing the permittivity from 25 to 100 as shown in the first row of FIG. 2 (at D). On the other hand, partial coverage of the sample with the modeled pads degraded the global B₁ transmit efficiency. However, using a curved pad demonstrated more efficiency than employing a flat patch (the second and third row in FIG. 2 (at D)). The maximum average SAR calculated in the reference case was 10.43 W/kg. FIG. 2 (at E) displays the average SAR for dielectric pads wrapped around (first row) and curved about the phantom (second row). The calculated average SAR values did not change significantly by using dielectric pads in any case. However, using dielectric pads reduced the measured maximum average SAR from 10.43 W/kg to 9.94 W/kg when a dielectric pad with εᵣ = 100 is wrapped around the sample.

The resonance frequency of the coil was measured as 299.62 MHz for the reference condition without any dielectric pad. When the whole sample was wrapped with a dielectric pad, the resonance frequency of the coil changed from 299.62 MHz to 296.68 MHz for εᵣ = 25, 294.57 MHz for εᵣ = 50, and 290.85 MHz for εᵣ = 100. Due to a significant shift in the resonance frequency at higher dielectric constants, commercial scanners with limited features to observe and correct the coil resonance would benefit from low εᵣ values of the proposed stretchable dielectric pads. The second and third row of FIG. 2 (at D) show that partially covering the phantom with a dielectric pad increases the peripheral field intensity closer to the patch but decreases the signal intensity at the center, compared to a no-pad condition as a reference in FIG. 2 (at B). However, wrapping the pad around the sample improves both central and peripheral field intensities along with homogeneity.

### Imaging

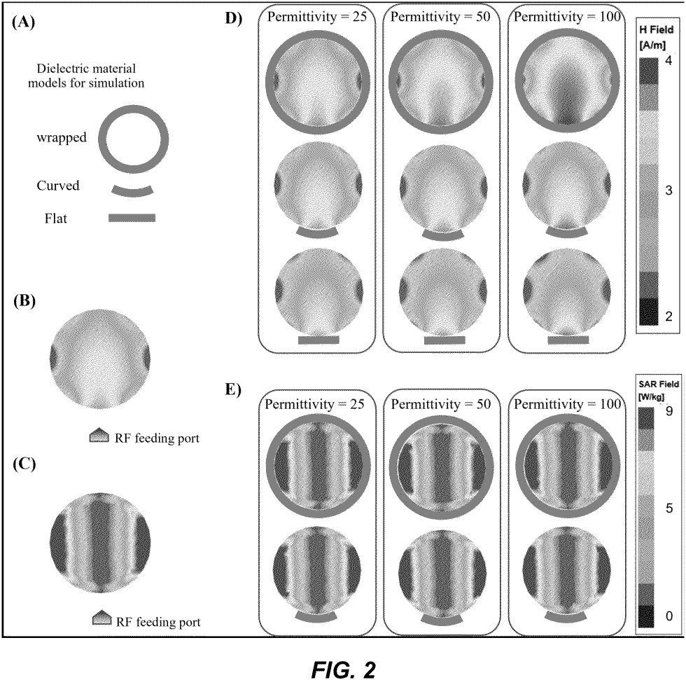

Three imaging experiments were conducted to evaluate the performance of the developed dielectric pads. In the noise analysis and MRI invisibility check, each pad was wrapped around the phantom multiple times, thus creating more potential noise sources, as shown in FIG. 3. The standard deviation of noise was measured in the empty space surrounding the small (2.85 cm diameter) cylindrical phantom in the four corners of each image and averaged. The measured image noise level (unitless) around the phantom was 303 without utilizing any dielectric pad (FIG. 3 (at A)), 307 with the solely elastic polymer (Ecoflex) pad (FIG. 3 (at B)), 232 with the wrapped BaTiO₃-based pad (FIG. 3 (at C)), and 260 with the wrapped TiO₂-based pad (FIG. 3 (at D)). A spatial offset was observed in FIG. 3 (at B) and 3 (at C). It demonstrates that the elastic polymer (Ecoflex) is a main source of the chemical shift artifacts. However, the TiO₂-based dielectric material in FIG. 3 (at D) and the SiC-based dielectric material in FIG. 3 (at E) can be invisible on MR images and eliminate the chemical shift artifact induced by the spatial offset between the pad and the phantom.

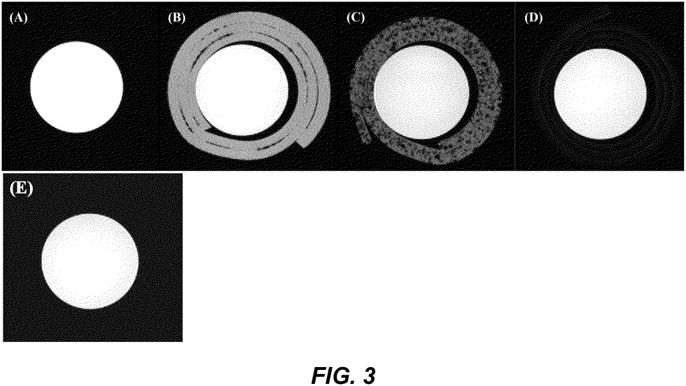

FIG. 4 shows the signal intensity and signal uniformity comparison of a completely covered (top of FIG. 4 (at A)), a partially covered (bottom of FIG. 4 (at A)), and an uncovered (FIG. 4 (at B)) isotropic phantom (5 cm diameter). Although enhancement in signal intensity is minor when wrapping the pure Ecoflex-based pad around the phantom (top of FIG. 4 (at C)), which comes from the small permittivity (εᵣ = 2.8) of this material, using TiO₂- and BaTiO₃-based dielectric pads, significantly increases the signal intensity and uniformity. An improvement of 33.81% and 44% in mean signal intensity, and 29.96% and 27.76% in signal uniformity was observed by completely wrapping the BaTiO₃-based (top of FIG. 4 (at D)) and TiO₂-based (top of FIG. 4 (at E)) pads around the phantom, respectively. Same pattern (except for Ecoflex) with less signal improvement compared to completely wrapped case is detectable in using the curved pads (bottom of FIG. 4 (at C, D, and E). FIG. 4 (at F and G) show a numerical demonstration of signal intensity versus distance in pixels along the dotted line in FIG. 4 (at A) for wrapping around the phantom and using curved patches on the phantom. The plots indicate that wrapping dielectric pads around the phantom improves the signal level more than simply using a curved patch.

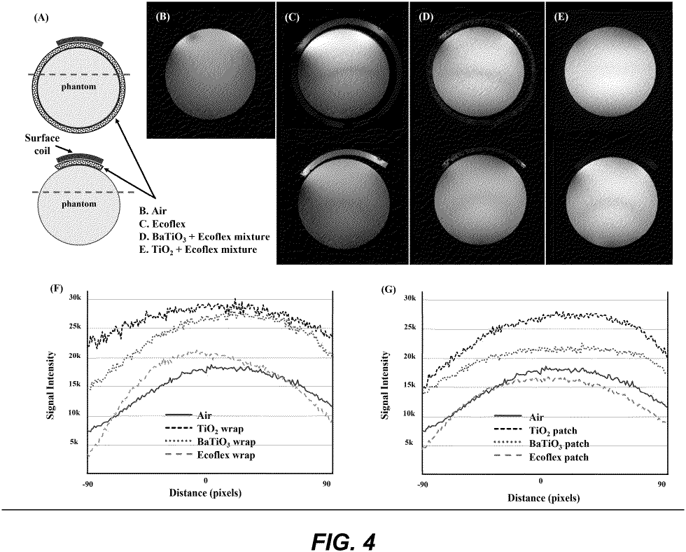

An oxtail sample was loaded into a cylindrical holder (5.8 cm diameter) and a pad was wrapped around the oxtail for MRI scans. SNR increased 8.74% and 7.23% with wrapping TiO₂- and BaTiO₃-based dielectric pads around the oxtail, respectively, whereas the improvement was only 0.66% with using the pure Ecoflex-based pad (FIG. 5 (at A, B, C, and D)). Oxtail images show chemical shift artifacts with the purely Ecoflex-based pad (FIG. 5 (at B)) and BaTiO₃-based pad (FIG. 5 (at C)). However, the TiO₂-based pad can be invisible in the image and eliminate the spatial offset from the chemical shift (FIG. 5 (at D)). These imaging results demonstrate that the mixture of dielectric powders and a silicone elastomer (Ecoflex) can increase B₁ field uniformity, intensify the MR signal level, and enhance the SNR of images with stretchable, flexible and MR invisible capabilities.

The optimal permittivity (εᵣ,ₒₚₜᵢₘₐᵣ) for an arbitrary operation frequency has been empirically derived, as shown in equation 2:

$${ε}_{r , o p t i m a l} \approx \frac{7 \times {10}^{7}}{{f}^{2.248}} \qquad \text{(Eq. 2)}$$

By substituting ¹H frequency at 7T field strength (~298 MHz) in the above equation, (ε_{r,optimal}) would be 191.89. However, during the simulations with dielectric pad around the phantom, large changes in dielectric constant were observed causing significant mismatch to the coil tuning and matching condition. Furthermore, this became a challenge to correct for when the MR console had no ability to display the resonance condition of the RF coil under test. This created a trade-off between large values of permittivity and change in resonance condition of the coil. The proposed dielectric pads may have permittivity of less than a few dozen, but they improved the transmit efficiency with little influence on the coil resonance. However, the lower dielectric constant was compensated for by fully wrapping the dielectric pad around the sample. Since the TiO₂ used for this study is anisotropic, the dielectric constant must be represented as dyadic product in matrix form. An effective dielectric constant of the mixture was calculated using the Maxwell-Garnett equation. The mixture contained an isotropic host medium with anisotropic inclusions, representing Ecoflex and TiO₂ respectively, where the anisotropic ellipsoidal inclusions degenerated into spheres. The assumption of an ordered mixture with spherical degeneration was convenient without being able to predict the ellipsoid orientation. If the ellipsoid orientation is known, then the depolarization factors implicit in the Maxwell-Garnett equation can be determined, thus the effective dielectric constant can be more rigorously predicted. But, for such a low volumetric filling factor of TiO₂, the increased accuracy would not necessarily be impactful on the study. Similarly, the Maxwell-Garnett equations in non-dyadic form can be used for the BaTiO₃ dielectric pads as well.

Similar signal intensity with the purely Ecoflex-based pad was anticipated and observed. Like other fabricated pads, wrapping Ecoflex around the sample performed better than using a small patch of it. Despite the performance of other pads, a small patch of just Ecoflex decreased the signal intensity compared to not having any pads. This phenomenon was predictable by comparing the simulation results of magnetic field distribution of the reference (FIG. 2 (at B)) without any pad with magnetic field distribution of a low dielectric constant (εᵣ = 25) small patch in the first column of FIG. 2 (at D). A strong signal from the Ecoflex may induce frequency shifts as the material is imaged.

A new invisible mixture composed of Ecoflex and high concentrations of TiO₂ powder was introduced. This finding opens the door for more experiments with the goal of using other dielectric materials such as silicon carbide (SiC) or silicon Nitride (Si₃N₄) for flexible and stretchable dielectric materials. The chemical shift artifact is a primary challenge. However, invisibility with the elimination of the unwanted chemical environment of precessing protons in the dielectric materials can mitigate or eliminate this problem. Also, the spatial offset can be estimated in advance and fixed by adjusting the receiver bandwidth per pixel and size of the frequency encoding matrix. Finding other biocompatible elastomers may be an alternative way to minimize this artifact by accommodating more dielectric powders for flexible and stretchable dielectric materials in MR imaging.

This study introduced a novel flexible, stretchable and MR invisible dielectric material that can increase MR signal intensity and uniformity. Imaging results that were consistent with those of the simulations showed an improvement of 44% and 27.76% in signal intensity and B₁ uniformity, respectively, by wrapping the pad around the entire circumference of the sample. Technical feasibility of dielectric elastomers had been validated with MR imaging in this study.

### Example 2 - Wearable Device to Optimize MRI Images

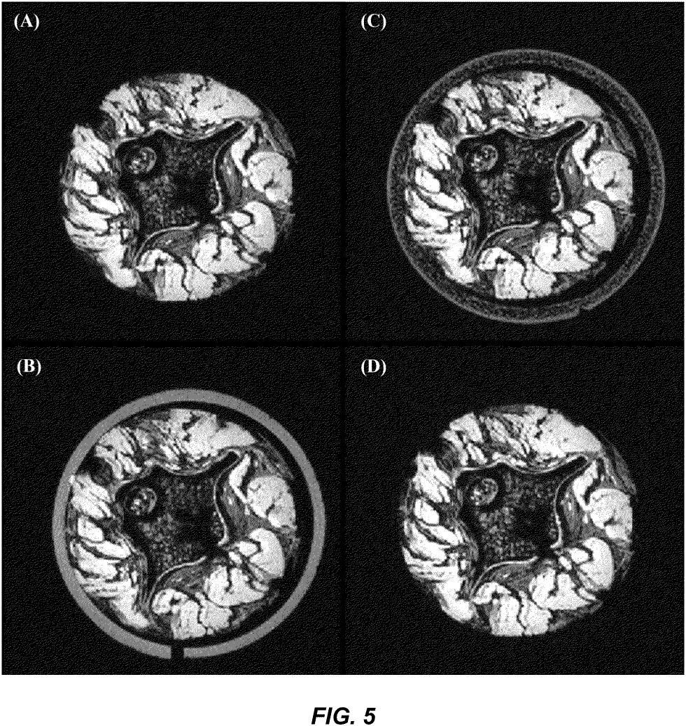

FIG. 6 (at a) shows that a higher dielectric constant is not always better, and a wrap-around shape may be better than a local pad with a reasonable dielectric constant level, neither too high nor too low. Dielectric helmets (in FIG. 6 (at b)) have shown promising results of field uniformity and SNR. However, the helmets are rigid in form and cannot adapt to various head sizes and shapes. The helmet increases the inhomogeneity in the RF field interference and overlap. Recently, artificial EM materials have been reported for MR imaging. However, bulky structures (in FIG. 6 (at c)) require a fairly large space, and the measurable MR signal significantly drops with the third power of the distance between an RF coil and a subject. The induced magnetic field (B₁) by a coil is defined as

$${B}_{1} = \frac{{μ}_{0} I {R}^{2}}{2 {\left({R}^{2} + {d}^{2}\right)}^{\frac{3}{2}}}$$

by the Biot-Savart Law, where, R is the radius of the coil, I is the magnitude of current flow and d is the distance between a coil and a subject, thus resulting in the significant drop.

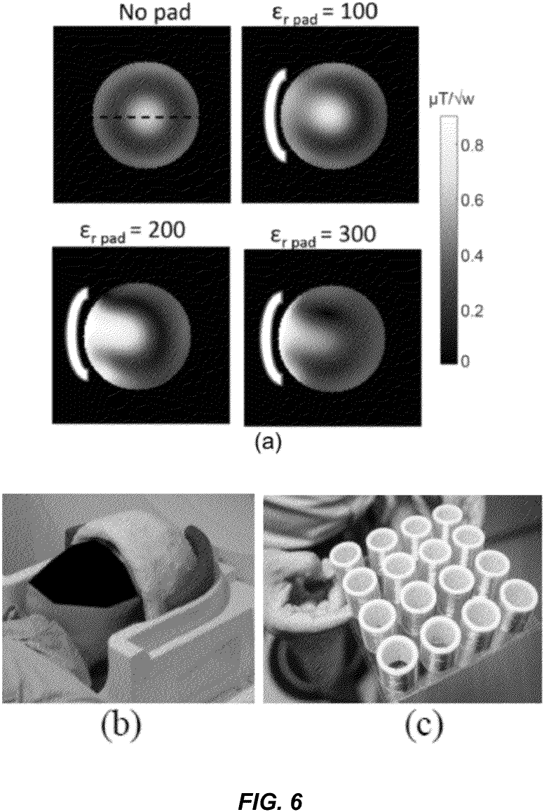

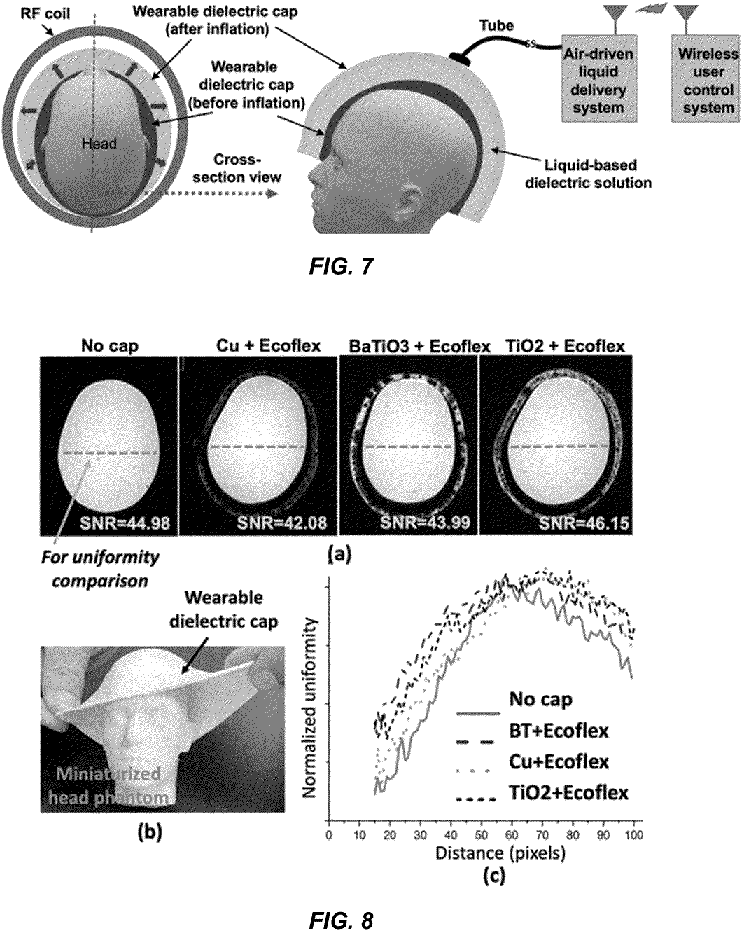

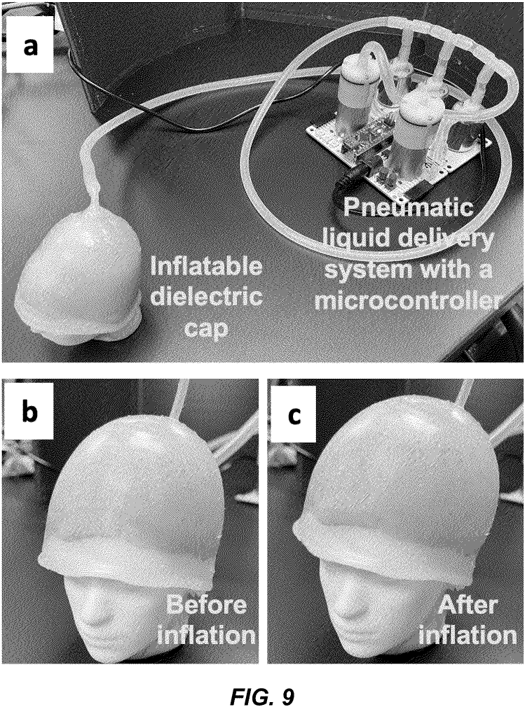

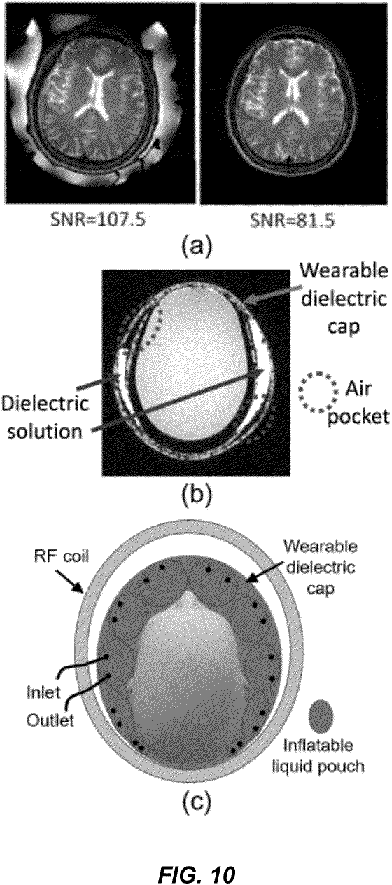

FIG. 7 illustrates a wearable, inflatable, and tunable dielectric cap 10 for human brain imaging according to an embodiment of the present disclosure. In one configuration, magnetic field strength of the MRI system is 7T, however other magnetic field strengths may be employed. For example, a wearable pad may be utilized with a 3T magnetic field strength MRI system. In other embodiments, the wearable pad may be utilized with a 1.5T magnetic field strength MRI system.

The cap 10 includes the material described above with respect to the dielectric pad. The material is used to fabricate a wearable dielectric cap that wraps around the human head with high elasticity and comfort for the user. A thin (e.g., 10 mm or less), tight-fitting wearable structure generates homogeneous RF boundary conditions between the cap and the head. The specific absorption rate (SAR) can be reduced with the use of dielectric materials. The wearable cap provides a dielectric material for clinical use with the improved B₁ field uniformity and signal-to-noise ratio (SNR) as well as reduced SAR at ultrahigh fields.

The cap 10 provides a tunable function. The cap 10 is wirelessly controlled by a user for optimizing the performance of the dielectric cap to provide better image quality. The cap 10 includes empty spaces, which are expanded or contracted using a liquid-based dielectric solution to adapt to various electrical and geometrical conditions encountered in clinical examinations and a wireless air-driven liquid delivery system as shown in FIG. 7.

The automatic feedback control system was developed with a pressure sensing and wireless control that monitors and adjusts the amount of the dielectric solution for patient comfort, immobilization, reduction of the leakage risk, and less RF interfaces. Such tunable function can be used for inflation/deflation of the cap 10 and for additional B₁ field adjustment with a wireless control during the scan, if necessary. In addition, such function may support the head and alleviate head motion artifacts.

Dielectric material mixtures for 7T MRI applications were optimized. A mixture of the silicone elastomer (e.g., ecoflex®) with barium titanate (BaTiO₃), titanium dioxide (TiO₂), or silicon carbide (SiC) particle had good performance in the preliminary study. Fabrication began with a stretchable form with Ecoflex, a platinum-catalyzed silicone and biocompatible rubber, and then other polymers were considered for the stretchable function. Additional high dielectric materials such as calcium titanate (CaTiO₃), graphene oxide, gold nanoparticles, carbon nanotube, poly (3,4-ethylene dioxythiophene)-poly (styrene sulfonate) (PEDOT-PSS), etc. were examined with different solvents and elastomers. In particular, the suitability of graphene oxide, which has a high dielectric constant and MRI compatibility was assessed as an emerging material in MR imaging. A dielectric probe with a network analyzer was used to measure the dielectric constant of the fabricated mixtures. The measured dielectric constant was used for the electromagnetic (EM) simulation (HFSS, Ansys) to estimate the B₁ field distribution and SAR with the power deposition in Watts per kilogram of a tissue model using a human head model. A custom-built RF coil tuned at 297 MHz and a magnetic field pickup probe was used to measure RF transmission characteristics (S₂₁) with and without dielectric materials. At least three dielectric mixtures were selected for the fabrication of head caps.

Three head-size dielectric caps were fabricated using 3D printed molds with the materials discussed above. The bubbles in the dielectric material may increase the nonlinearity of RF propagation, so the fabrication process was developed for a high-quality mixture using a vacuum chamber and heat treatment technique. Since the current flow within a dielectric material induced by an RF transmitter has a strong relationship with the B₁ field distribution and SAR, the EM simulation was performed with a commercial 7T coil model and a human head model, and the results were compared with MR imaging. Reducing SAR was one of the benefits of the use of dielectric materials, so the reduced SAR with the wearable dielectric caps was demonstrated. Since a gap between a head and a wearable cap because of ears was observed in the preliminary study, the cap shape was designed for a tight-fitting form and wearing comfort. It increased the homogeneity of RF boundary conditions. For evaluation of safety, fiber optic temperature sensors and an infrared (IR) thermal camera with a 100-watt RF power amplifier were used to measure real temperature rise in the dielectric caps and phantoms.

Since the dielectric cap may contain ferro- or paramagnetic particles, they may cause magnetic susceptibility effects. Gradient echo imaging and fast spin echo images were obtained using the same experimental setup to emphasize any magnetic susceptibility effects caused by the dielectric caps. Two approaches were used for imaging tests: (1) using an on-campus preclinical 7T (17 cm bore-size, MDRYMAG9417, MR solutions) and (2) using the first FDA-approved whole-body 7T scanner. As shown in the preliminary results, the B₁ field uniformity was checked with isotropic phantoms despite a small bore and sample size allowed by the preclinical scanner. An isotropic phantom compared to anisotropic anatomy offer a better way to evaluate the uniformity and SNR. Thus, the preclinical scanner was used for choosing dielectric materials. The whole-body scanner was used to evaluate the uniformity, SNR, temperature change with a head size phantom for the fabricated caps. The B₁⁺ field uniformity at 298 MHz was demonstrated in three orthogonal planes with and without the dielectric cap using fast spin echo sequences. The fiber optic sensors within the head phantom measure the real temperature rising during the scan, and the results were compared with the SAR estimation.

An evaluation matrix among the RF transmission (S₂₁), B₁ field uniformity, SNR, SAR, and temperature with different thicknesses (< 10 mm), mixtures, and concentrations of the dielectric materials was generated for choosing the most effective three dielectric materials using the preclinical scanner. Using a head size phantom at a whole-body 7T, the assessment elements were uniformity, SNR, and temperature change. Horizontal and vertical lines within an image were used to plot the B₁ field distribution for the uniformity comparison. The SNR was measured with two ROIs in two separate regions employing the relative standard deviation of signal or noise. Because the thermal criteria most directly target the safety issue and local temperature can be directly measured, it was prudent to validate temperature models by direct temperature measurement using accurate phantoms. The temperature was measured on the predetermined depths and planes in the radial direction using fiber optic sensors within the head phantom. A data set was made of temperature rise versus input power to the coil for 3 hours (1 hour before RF pulses, 1 hour during the RF power deposition, and 1 hour after RF pulses) at least two times. This data was compared with the SAR measured in the EM simulation. The isotropic electric field probes were used to directly measure the electric field for further safety evolution. With the results of the assessment, the materials of the three dielectric caps were verified. The wearable caps may have more than 30% improvement in the B₁ field uniformity using the standard deviation comparison and more than 10% improvement in the SNR with the same RF input power compared to when any pads do not exist.

Pieces or slots can be added to the dielectric cap for a balanced and predictable current flow resulting in the uniform B₁ field distribution with the improved SNR if the induced current displacement within a dielectric cap has a strong shorted wavelength effect and influences B₁ field inhomogeneity.

The use of water pads as a dielectric material can improve the uniformity and SNR as shown in FIG. 5 (at a). MR imaging tests were performed with a water-based dielectric solution, which was a mixture of deionized water and copper powder inside the elastomer cap (shown in FIG. 4). Higher B₁ field intensity was measured with the dielectric liquid compared to the result of the powdered form as shown in FIG. 5 (at b). Empty spaces within the wearable dielectric cap were designed using 3D printed molds and multilayer bonding. The same dielectric particles were used for both the wearable form and liquid-based solution to reduce the heterogeneous RF boundary conditions. The air pockets (dotted red circle) were observed inside the cap with the dielectric solution. To solve this problem, the wearable dielectric cap had multiple pouches (in FIG. 5 (at c)) that contained a dielectric solution. Each pouch was connected to an individual control unit with an inlet and outlet tube. The Ecoflex has high elasticity and durability (tensile strength < 300 psi and elongation at break > 600%), and there are various types, so Ecoflex products with expansive force, tensile strength, elongation, modulus expansion, etc. were investigated for use in fabricating a reliable tunable cap.

In the preliminary study (shown in FIG. 4), an open-source hardware kit (e.g., available from Programmable-Air) was used with a wireless microcontroller module and lab-built interface circuits for controlling an inflatable dielectric cap. A wireless and automatic control system was constructed to implement the tuning function. After loading a subject in a coil, the inflatable function started, and a dielectric solution expanded the pouch. A pressure sensor informed the status of expansion and contraction, and the automatic control stopped the injection of the dielectric solution to prevent high pressure inside a pouch. The control unit was individually connected to each pouch and wirelessly controlled by a user. The interrelation between the volume of the dielectric solution and the performance of an RF coil was investigated due to high coupling. It was demonstrated that a balance could be found for a tunable range with expansion and contraction with the effect in RF characteristics (tuning and matching).

The same procedures and methods described above with the tunable function and supporting control system were employed. In addition, the SNR and uniformity were examined by the volume change of the dielectric solution with a head phantom. The ratio between the center of the head and the mean of the four or eight voxels around the brain served as a relative measure between the developed wearable dielectric cap and current available commercial pads with fast spin echo sequences. The electronics for the pneumatic control system can be placed outside the shielded magnet room and use long tubes to prevent any electronic noises in images during the scan.

In addition to the evaluation matrix and methods described above, the resonance frequency shifts by the volume change of the dielectric solution were added to the assessment elements to check the effect of the tunable function. Analysis of K-space data was used to check image artifacts from the dielectric solutions and control system. For assessing safety, the temperature change was less than 1° C. or local heating was less than 38° C. in the head phantom to comply with the FDA guidelines. Durability and reliability were ensured for long-term use. In particular, the use of dielectric solution with tunable function was evaluated for leakage, deformation, particle precipitation, and asymmetric solution distribution. A tensile stretching test was performed more than 1,000 times using an actuator and pressor sensor to confirm material integrity.

A liquid-based dielectric solution may present a leak issue. To address, a medical-grade silicone rubber balloon (e.g., endorectal balloon) was added for encapsulating the wearable cap, and a hydrogel was investigated that is one of the hydrophilic polymers that can swell in water and hold a large amount of water while maintaining structure in a gel type. If necessary, a liquid-based solution can be replaced with a hydrogel-based solution with a dielectric particle. Since the presence of a liquid solution influenced the resonance frequency shift but the amount of the liquid solution slightly changed the frequency in the preliminary results, the offset was discovered of the shifted resonance frequency and reflect it to RF coil settings.

In-vivo imaging tests were performed using a rat with different dielectric particles, solvents, and fabrication methods. In parallel with using isotropic phantoms, rat experiments with dielectric materials provided in-vivo imaging data to analyze potential artifacts by irregular magnetic susceptibilities and dielectric constants of tissues and anatomical structures. The comparison analysis between isotropic phantom results and in-vivo imaging results were examined. Such analysis was reflected in the EM simulation model to increase the accuracy and conformity of results between the simulation and imaging test.

Six human subjects were recruited for testing with the whole-body 7T scanner. The overall SNR and B₁ field uniformity using the selected three dielectric caps were examined with a commercial RF coil to compare the performance with a commercial pad and without a dielectric pad. Magnetic resonance thermometry (MRT) was used for further safety evaluation with the subjects. Magnetic susceptibility artifacts surrounding the head by the tight-fitting form and immeasurable dielectric effects by the actual head structure with isotropic phantoms was investigated. These artifacts can be readily visualized with dielectric particles, air, or water-based solution under ultrahigh magnetic fields. Gradient recalled echo imaging was used with relatively long TE values to check the artifacts. Since the use of shortened echo time pulses such as fast spin echo sequence can alleviate B₁ field inhomogeneity by dielectric effects, the field uniformity comparison with the dielectric caps was performed with fast spin echo and other long echo time pulses. Then, the results were compared to evaluate the performance of the wearable dielectric cap.

The assessment elements of in-vivo imaging tests were the B₁ field uniformity, SNR, feedback from subjects with and without the wearable dielectric cap. The uniformity and SNR using the methods described above were evaluated. The subjects were surveyed to examine any differences with and without the dielectric cap and to determine wearing comfort during the scan. The results from imaging tests and subject feedback from subjects established guidelines for clinical use of the wearable and tunable dielectric cap.

### Preliminary Data

Preliminary data shows a proof of concept and the potential problems to be solved in this project. Fifteen samples were made with different dielectric particles and solvents. RF signal transmission (S₂₁) penetrating the samples from an RF coil tuned at 297 MHz were measured to a pickup probe using a network analyzer. The bench test results showed that barium titanate (BaTiO₃), titanium dioxide (TiO₂), Copper (Cu) nano powder with Ecoflex (biocompatible rubber) had good RF transmissions. With this result, a few tight-fitting wearable dielectric caps were fabricated and imaging tests were performed with a miniaturized head phantom (in FIG. 3 (at b)) to fit a preclinical 7T for rapid results. MR images in FIG. 3 (at a) show that a wearable cap with the mixture of TiO₂ and Ecoflex increased the overall SNR from 44.98 to 46.15. For the B₁ field uniformity comparison, the normalized signal intensities along the dotted line in FIG. 3 (at a) are compared with and without dielectric caps. Most importantly, all dielectric caps improved the uniformity (in FIG. 3 (at c)) despite a preclinical 7T that has less effect of the shortened wavelength. The wearable dielectric cap with the optimized material and improved fabrication process improved the field uniformity and SNR in a whole-body 7T MRI.

A wearable dielectric cap was fabricated with a space that was filled by a water-based copper nanoparticle solution as shown in FIG. 4. A microcontroller with a wireless communication module, pneumatic liquid pumps, and lab-built interface circuits were employed for the expansion or contraction test of the dielectric cap (in FIG. 4 (at b) and 4 (at c)). Two different methods for liquid delivery were tested: a linear actuator with a syringe and an air-driven liquid pump. An actuator or air-driven pump with electrical circuits is not necessary to place next to a dielectric cap and long tubes can be used, so non-magnetic property in the control system is not mandatory. In some embodiments, there may be a non-magnetic property in the control system. In other embodiments, there may not be a non-magnetic property in the control system.

What is claimed is:

1. A dielectric pad comprising:
an elastomeric material; and
a dielectric material dispersed in the elastomeric material, the dielectric material comprising TiO₂, BaTiO₃, or SiC, or a combination thereof, wherein the dielectric pad has a dielectric constant of at least 10.

2. The dielectric pad of claim 1, wherein the dielectric material is TiO₂, and wherein a concentration of the TiO₂ is 20% by weight.

3. The dielectric pad of claim 1, wherein the dielectric material is BaTiO₃, and wherein a concentration of the BaTiO₃ is 55% by weight.

4. The dielectric pad of claim 1, wherein the dielectric material is SiC, and wherein a concentration of the SiC is 58% by weight.

5. A method of fabricating a dielectric pad for magnetic resonance imaging, the method comprising:
mixing a dielectric material with an elastomer to form a solution;
pouring the solution into a mold;
applying a vacuum to the solution to remove air bubbles; and
maintaining the solution at room temperature.

6. The method of claim 4, further comprising placing the mold into an oven for curing.

7. The method of claim 4, wherein applying the vacuum occurs for about 5 minutes.

8. The method of claim 6, wherein the solution remains at room temperature for about 10 minutes.

9. A device to optimize images of a target acquired by MRI scanner, the device comprising:
a cap wearable on a head, the cap comprising a mixture of a dielectric material and an elastomeric material, the cap configured to inflate and deflate to optimize MR images acquired by the MRI scanner.

10. The device of claim 9, wherein the dielectric material comprises BaTiO₃, TiO₂, or SiC.

11. The device of claim 9, wherein the dielectric material comprises copper nano powder.

12. The device of claim 9, wherein the cap has a dielectric constant of at least 10.

13. The device of claim 9, wherein the cap includes a thickness between about 1 mm to about 10 mm.

14. The device of claim 9, wherein the cap includes a plurality of cavities in communication with a liquid delivery system.

15. The device of claim 14, wherein the liquid delivery system provides a liquid-based dielectric solution to the cavity for the cap to adapt to electrical and geometrical conditions to optimize the MR images.

16. The device of claim 15, wherein the liquid-based dielectric solution comprises a water-based dielectric solution.

17. The device of claim 15, wherein the liquid-based dielectric solution and the dielectric material comprise the same dielectric material.

18. The device of claim 14, wherein each cavity includes an inlet and an outlet in communication with a dedicated liquid delivery system.

19. The device of claim 9, wherein the MRI scanner is a 3T MRI scanner.

20. The device of claim 9, wherein the MRI scanner is a 7T MRI scanner.
